(19) United States
(12) Patent Application Publication
(10) Pub. No.: US 2005/0038323 A1
(43) Pub. Date: Feb. 17, 2005

(54) Tear duct endoscope for medication and sampling

(75) Inventor: Carlo Maria Valazzi, Pesaro (IT)

Correspondence Address: Geoffrey R. Myers, Esquire;Hall, Priddy, Myers & Vande Sande, Ste. 200, 10220 River road, Potomac, MD, 20854, US

(73) Assignee: Knit Ventures, LLC

(21) Appl. No.: 10/637,707

(22) Filed: Aug. 11, 2003

(51) Int. Cl. A61B 1/015

(52) U.S. Cl. 600/153

(57) ABSTRACT

An endoscope for medication and sampling, having a grip portion; and a cannula connected at one end to the grip portion, and in which are formed a first conduit housing optical diagnosis means, and a second conduit outside and coaxial with the first conduit and having three lateral holes. The second conduit is for the passage of medicinal substances, or for withdrawing sample material by suction.

The present invention relates to a tear duct endoscope.

### BACKGROUND OF THE INVENTION

Over the past twenty years, endoscopic techniques have become widely used in diagnosing and treating tear duct problems.

Endoscopic techniques permit intervention inside the tear duct, through the opening of the duct itself, as opposed to operating surgically from the outside.

Endoscopy as related to tear ducts employs an endoscope comprising a cannula, which is inserted inside the tear duct, and in which is formed a channel housing optical fibres for illuminating and visualizing, as is known, the interior of the tear duct, to permit non-invasive inspection of the condition of the duct, e.g. the existence and location of occlusions or stenoses, and simplify diagnosis of the problem involved.

In the latest embodiments, tear duct endoscopes are designed, either separately or in combination, to flush the channel with water to enhance the images obtained by the optical fibres, and the cannula is fitted on its free end with a cutting member or, at any rate, a member capable of mechanically removing occlusions or stenoses.

A major drawback of endoscopes of the above type lies in their only visualizing the tear duct during diagnosis and/or mechanical removal of the obstruction, whereas confined medication and/or histological sampling are performed with no possibility of visualizing the work area.

### SUMMARY OF THE INVENTION

It is an object of the present invention to provide an endoscope designed to permit confined medication and/or sampling.

According to the present invention, there is provided an endoscope for medication and sampling, comprising a grip portion; and a cannula connected at one end to said grip portion, and in which is formed a first conduit housing optical diagnosis means; said endoscope being characterized in that a second conduit, having at least one outlet and separated hermetically from said first conduit, is formed in said cannula.

In a preferred embodiment of the endoscope according to the invention, said first conduit is located inside and coaxially with said second conduit.

The endoscope as described above has the big advantage of enabling effective medication and/or sampling inside the tear duct by simultaneously visualizing the interior of the duct.

### BRIEF DESCRIPTION OF THE DRAWINGS

A non-limiting embodiment of the invention will be described by way of example with reference to the accompanying drawings, in which:

FIG. 1 shows a partial longitudinal section of a preferred embodiment of the endoscope according to the present invention;

FIG. 2 shows a larger-scale detail of FIG. 1;

FIG. 3 shows a further larger-scale detail of FIG. 1;

FIG. 4 shows a section, with parts removed for clarity, along line IV-IV in FIG. 3.

### DETAILED DESCRIPTION OF THE INVENTION

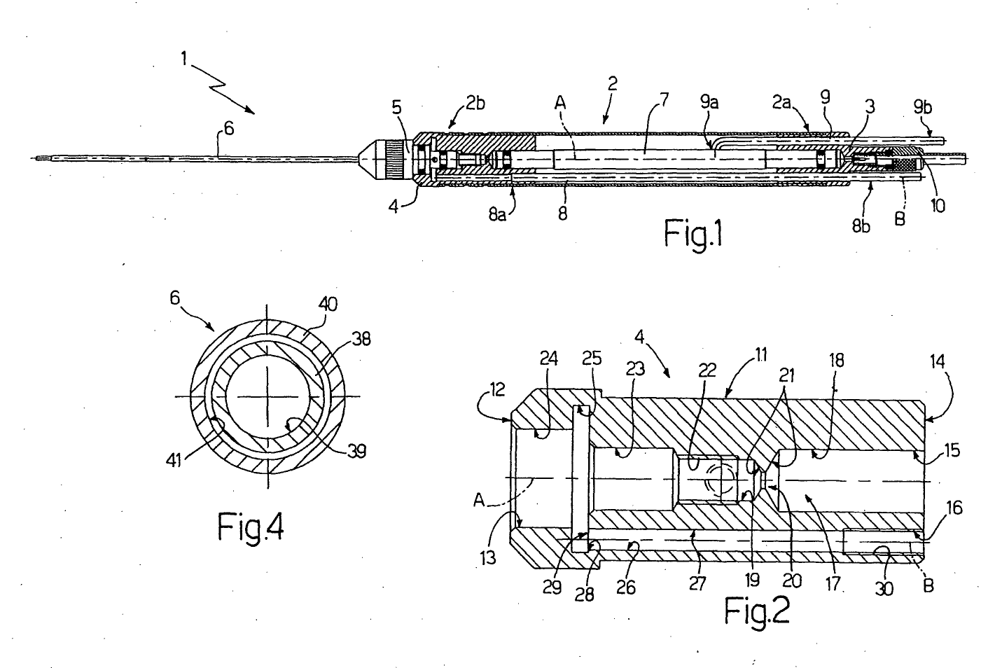

Number 1 in FIG. 1 indicates as a whole the endoscope according to the present invention.

Endoscope 1 comprises a cylindrical grip portion 2 of axis A; a rear connecting portion 3 partly engaged inside a rear end 2a of grip portion 2; a front connecting portion 4 partly engaged inside a front end 2b of grip portion 2; and a cannula-holder 5 engaging front portion 4 and to which a cannula 6 is welded. Rear portion 3 and front portion 4 are fixed to grip portion 2 by known fastening means not described.

Grip portion 2 houses a first tube 7, coaxial with axis A, for the passage of the optical fibres and flush liquid, and the ends of which engage rear portion 3 and front portion 4 respectively.

Grip portion 2 also partly houses a second tube 8 for the passage of medicinal substances or material for sampling, and a third tube 9 for topping up the flush liquid in first tube 7. Second tube 8 has an axis B parallel to axis A, extends through rear portion 3, and has a first end 8a inside grip portion 2 and engaging front portion 4, and a second end 8b outside grip portion 2. Third tube 9 extends through rear portion 3, and has a first end 9a inside grip portion 2 and connected to first tube 7, and a second end 9b outside grip portion 2.

Rear portion 3 is substantially cylindrical, and comprises three through holes, two of which are engaged, in use, by second and third tube 8 e 9 respectively, while the third hole is partly engaged by first tube 7.

As with other endoscopes of the same type as the one according to the invention, rear portion 3 is connected externally to a fitting 10 for correct insertion and positioning of the optical fibres, which, as will be seen, are housed inside first tube 7 and extend through the rest of device 1 up to the free end of cannula 6 to visualize, as is known, the interior of the tear duct into which cannula 6 is inserted.

As shown in FIG. 2, front portion 4 is symmetrical with respect to axis A, and is defined by a substantially cylindrical outer lateral surface 11; by a first end surface 12 perpendicular to axis A, and in which is formed a circular opening 13 of axis A; and by a second end surface 14 perpendicular to axis A, and in which are formed a first circular opening 15 of axis A, and a second circular opening 16 of axis B.

Front portion 4 has a first through hole 17 coaxial with axis A and defined, as of circular opening 15, by a first cylindrical inner surface 18 housing, in use, one end of first tube 7; and by a second cylindrical inner surface 19 smaller in diameter than first cylindrical inner surface 18, and separated from it by a constriction 20 defined by two tapering surfaces 21, each extending from cylindrical inner surface 18 and cylindrical inner surface 19. A thread 22 is formed on cylindrical inner surface 19, and is engaged, in use, by a portion of cannula-holder, as described later on. Downstream from cylindrical inner surface 19, through hole 17 is defined by a third cylindrical inner surface 23 larger in diameter than cylindrical surface 19; and by a fourth cylindrical inner surface 24 larger in diameter than cylindrical inner surface 23, and which terminates at circular opening 13 in end surface 12. An annular groove 25 is formed between cylindrical inner surfaces 23 and 24, defines a connecting chamber, and is larger in diameter than cylindrical inner surface 24.

Front portion 4 has a second hole 26, of axis B, defined by a cylindrical inner surface 27, extending from circular opening 16 in end surface 14 to annular groove 25, and communicating with annular groove 25 through an opening 28 formed in an annular radial surface 29 of annular groove 25. More specifically, cylindrical inner surface 27 comprises a threaded portion 30 located close to circular opening 16 in end surface 14, and engaged, in use, by end 8a of second tube 8.

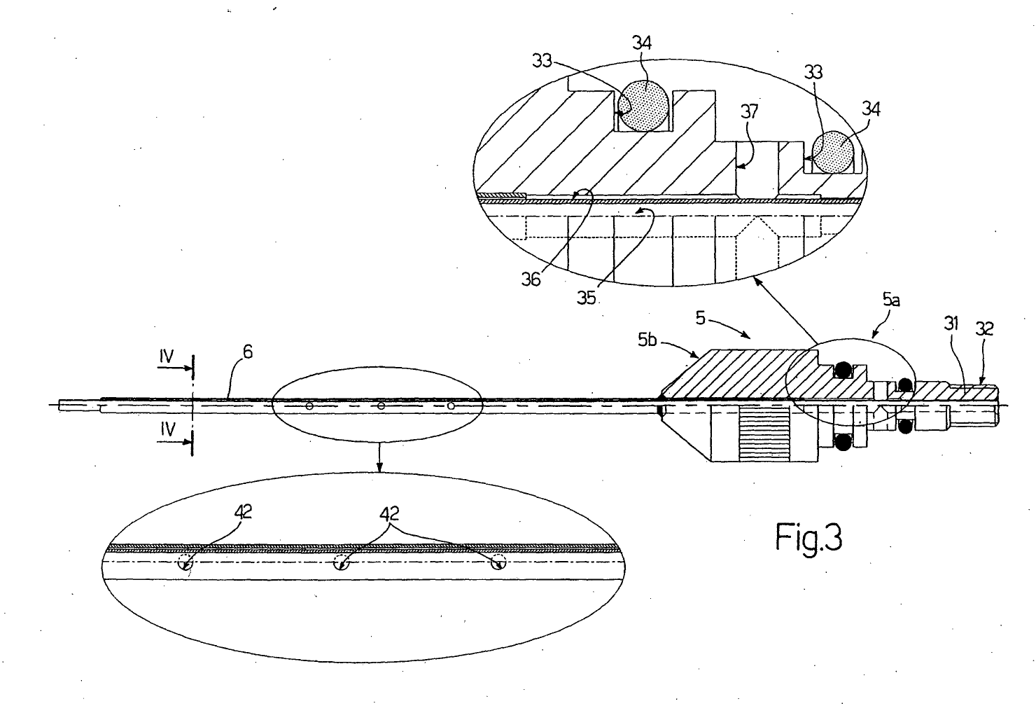

As shown in FIG. 3, cannula-holder 5 is symmetrical with respect to axis A, and comprises a rear portion 5a designed to partly engage hole 17 in front portion 4 through circular opening 13 in end surface 12; and a front portion 5b positioned, in use, outside front portion 4. Cannula-holder 5 is fixed to front portion 4 by a cylindrical end portion 31, the outer surface 32 of which is threaded and engages thread 22 on cylindrical inner surface 19. Two circular grooves 33 are formed in an outer wall of rear portion 5a, and house respective sealing rings 34, which are forced, in use, against respective cylindrical inner surfaces 23 and 24 of through hole 17 in front portion 4.

A through conduit 35, of axis A, is formed in cannula-holder 5, is connected, in use, to first tube 7, and therefore houses the optical fibres and flush liquid. A cylindrical gap is formed about through conduit 35 in cannula-holder 5, and defines an annular-section conduit 36 extending about the whole of, and coaxial with, through conduit 35. Annular-section conduit 36 occupies the whole of front portion 5b, and only part of rear portion 5a, and is cut off at the sealing ring 34 closer to cylindrical end portion 31. More specifically, annular-section conduit 36 communicates with annular groove 25 in front portion 4 via two radial holes 37 formed in cannula-holder 5, and which permit passage of medicinal substances into conduit 36, and of sample material into annular groove 25. That is, medicinal substances travel along second tube 8 into hole 26 in front portion 4, then through opening 28 into annular groove 25, and through holes 37 into conduit 36; and the organic sample material follows the same path in reverse.

Finally, cannula 6 is welded to front portion 5b of cannula-holder 5, and is defined by an inner cylindrical wall 38 defining a conduit 39 of axis A; and by an outer cylindrical wall 40 coaxial with inner cylindrical wall 38, and defining with it an annular-section conduit 41 (FIG. 4). In other words, conduits 39 and 41 are extensions of respective conduits 36 and 35 to permit passage of the optical fibres and flush liquid into conduit 39, and of the medicinal substances or sample material into conduit 41. More specifically, conduit 41 is closed axially at its free end, and communicates externally via three lateral holes 42, by which to deposit medicinal substances or take samples by suction in a confined region of the tear duct.

Conduit 41 is closed axially to keep out other liquids or substances which may possibly contaminate the medicinal substances or, worse still, the sample material for analysis.

In actual use, after connecting tube 8 to a medication supply circuit or suction system, connecting tube 9 to a flush liquid supply circuit, inserting the optical fibres inside the whole device, i.e. up to the end of cannula 6, and connecting the appropriate endoscopic instruments, the cannula is inserted inside the tear duct in known manner to observe the interior of the tear duct on a screen and determine, on the screen, the precise region in the tear duct in which to deposit the medicinal substance or take the sample.

As will be clear from the foregoing description, the endoscope according to the invention has the big advantage of permitting medication or sampling while simultaneously viewing the area concerned by means of optical fibres.

1) An endoscope for medication and sampling, comprising a grip portion; and a cannula connected at one end to said grip portion, and in which is formed a first conduit housing optical diagnosis means; said endoscope being characterized in that a second conduit, having at least one outlet and separated hermetically from said first conduit, is formed in said cannula.

2) An endoscope as claimed in claim 1, characterized in that said first conduit is located inside and coaxially with said second conduit.

3) An endoscope as claimed in claim 2, characterized by comprising a cannula-holder between said grip portion and said cannula; said first and said second conduit extending inside said cannula-holder.

4) An endoscope as claimed in claim 3, characterized by comprising a first tube connected to said first conduit, and a second tube connected to said second conduit; said first and said second tube being housed inside said grip portion.

5) An endoscope as claimed in claim 4, characterized in that said first tube is coaxial with said first conduit.

6) An endoscope as claimed in claim 4, characterized in that said grip portion comprises a front connecting member connected to said cannula-holder.

7) An endoscope as claimed in claim 6, characterized in that the second tube and said second conduit communicate via a chamber formed in said front connecting member.

8) An endoscope as claimed in claim 4, characterized by comprising a third tube housed in said grip portion and having one end connected to said first tube.

9) An endoscope as claimed in claim 8, characterized in that said second and said third tube each have a portion outside said grip portion.

10) An endoscope as claimed in claim 1, characterized in that said second conduit has at least one lateral opening formed in said cannula, and by which to dispense medicinal substances or withdraw sample material by suction.

11) An endoscope as claimed in claim 10, characterized in that said lateral opening is defined by three holes.
